SEQUENCE LISTING (1 sequence)

SEQ ID NO: 1 - protein, 561 aa, Artificial Sequence
  CRM197 Protein
MGSSHHHHHHSSGLVPRGSHMDDDDKGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

(19) United States
(12) Patent Application Publication
(10) Pub. No.: US 2021/0032284 A1
(43) Pub. Date: Feb. 4, 2021

(54) METHOD FOR EFFICIENTLY RECOVERING AND PURIFYING ACTIVE CRM197 FROM INSOLUBLE CRM197 PROTEIN EXPRESSED IN INCLUSION BODY

(71) Applicant: FORBIOKOREA CO., LTD., Seoul (KR)

(72) Inventors: Hyeon Cheol LEE, Namyangju-si, Gyeonggi-do (KR); Bong Seong KOO, Seoul (KR); Hyoung Jong SEO, Seoul (KR); Jin Sook KIM, Gwangmyeong-si, Gyeonggi-do (KR); Ah Reum PARK, Pyeongtaek-si, Gyeonggi-do (KR); Seung Won JANG, Uijeongbu-si, Gyeonggi-do (KR)

(73) Assignee: FORBIOKOREA CO., LTD., Seoul (KR)

(21) Appl. No.: 16/965,743

(22) PCT Filed: Sep. 10, 2018
(86) PCT No.: PCT/KR2018/010541

§ 371 Date: Jul. 29, 2020

(30) Foreign Application Priority Data
Feb. 2, 2018 (KR) 10-2018-0013207

(51) Int. Cl. C07K 1/113; C07K 1/16; C07K 14/34

(52) U.S. Cl. CPC C07K 1/113; C07K 14/34; C07K 1/16

(57) ABSTRACT

Disclosed is a method for expressing and purifying insoluble proteins of CRM197. The method includes: culturing a transformant transformed with an expression vector containing a gene encoding CRM197 protein; obtaining a culture of the transformant and disrupting the cell to recover an inclusion body; solubilizing the inclusion body; treating the solubilized inclusion body with a refolding reaction mixture and performing a refolding process; and purifying the refolded CRM197 protein by chromatography.

### CROSS-REFERENCE TO PRIOR APPLICATIONS

This application is a National Stage Patent Application of PCT International Patent Application No. PCT/KR2018/010541 (filed on Sep. 10, 2018) under 35 U.S.C. § 371, which claims priority to Korean Patent Application No. 10-2018-0013207 (filed on Feb. 2, 2018), which are all hereby incorporated by reference in their entirety.

### BACKGROUND

The present invention relates to a method for expressing and purifying insoluble CRM197 protein.

Diphtheria toxin (DT) had been one of the major respiratory diseases causing outbreaks in children. The CRM197 protein is a variant of DT (58 kDa) characterized by a single mutation (G52E substitution). Recently, CRM197 was shown to have reduced toxicity while retaining the same inflammatory and immune-stimulant properties as diphtheria toxin, allowing it to be used as a safe carrier antigen in conjugated vaccines. The CRM197 can be covalently linked to poorly immunogenic and T-cell-independent capsular polysaccharides as carrier for vaccines, and the resulting conjugated vaccines is known to create efficiently T-cell-dependent conjugate antigens that are highly immunogenic in infants. Like the wild-type toxin, CRM197 comprises two domains, fragment A (catalytic) and fragment B, bonded together by a disulfide bridge. The B domain contains one subdomain for the binding to the HB-EGF cell receptor and another subdomain for translocation inside the cell. Recently, CRM197 was produced as a single peptide where fragment A was linked to fragment B easily forming the disulfide bond between them. Additionally, in addition to its vaccine adjuvant properties, there is growing interest in CRM197 because of its potential antitumor activity related to its ability to bind the soluble form of HB-EGF, which is highly expressed in some human cancers.

In initial developmental studies, CRM197 and other non-toxic variants were produced using lysogenic cultures of Corynebacterium diphtheriae, infected by particular β phages whose genome contains a mutated version of the tox gene encoding the DT. Several approaches utilizing alternative hosts were also studied, due to difficult operating conditions and low yields from fermentation using C. diphtheriae. Several attempts have also been conducted in Escherichia coli. However, expression of recombinant CRM197 was limited due to formation of inclusion bodies caused by reductive cytoplasmic expression. To overcome this problem, the expression of recombinant CRM197 was attempted in Bacillus subtilis using the subtilisin signal sequence for secretion into the culture medium. However, the maximum yield obtained was about 7.1 mg/L. Secretion can be of value in decreasing the costs of protein recovery. Likewise, in E. coli, the secretion of CRM197 protein to the periplasmic space has been considered as possible approach to decrease the costs of protein recovery. In an effort to find an additional host for expression of CRM197 that might spawn similar technology development, Pfenex co. ltd. turned their attention to a Pseudomonas fluorescens expression system. Recently, the production of soluble CRM197, comparable to E. coli-based expression, was achieved using P. fluorescens, which was routinely optimized in 20-L fermenters with a yield of 1-2 g/L.

Even though efficient expression of CRM197 was achieved in several microorganisms, denaturation/refolding of inclusion bodies by denaturing solubilization agents i.e. guanidine hydrochloride (GdnHCl) or urea, were still required. However, from an economic standpoint, this is not desirable for industrial processes due to poor recovery of functional protein. Recently, alternative methods for recovery of insoluble proteins expressed in E. coli were reported. Compared to traditional denaturation/refolding, these methods use non-denaturing solubilization agents and could obtain higher recovery yields for native protein, depending on properties of each protein. These mild solubilization agents retain the existing secondary structures of proteins to some extent and inhibit protein aggregation during refolding, resulting in improved recovery of bioactive proteins.

Against this backdrop, there is a need for methods to produce CRM197 in E. coli in an efficient and cost-effective manner.

### SUMMARY

The present invention provides a method for expressing and purifying bioactive CRM197 with high yield by solubilizing insoluble CRM197 proteins expressed as inclusion bodies in Escherichia coli with a non-denaturing solubilization agent and purifying the CRM197 proteins by Ni-affinity chromatography.

In order to solve the above problem, the present invention provides a method for producing a recombinant CRM197 protein, the method comprising: (a) a step for culturing a transformant transformed with an expression vector containing a gene encoding CRM197 protein; (b) a step for obtaining a culture of the transformant and disrupting the cell to recover an inclusion body; (c) a step for solubilizing the inclusion body; (d) a step for treating the solubilized inclusion body with a refolding reaction mixture and performing a refolding process; and (e) a step for purifying the refolded CRM197 protein by chromatography.

The present invention also provides the method for producing a recombinant CRM197 protein, wherein the solubilization is performed by a non-denaturing solubilization method.

In addition, the present invention provides the method for producing a recombinant CRM197 protein, wherein the non-denaturing solubilization method uses Tris-Cl buffer, DMSO, n-propanol or sarkosyl solution.

Furthermore, the present invention provides the method for producing a recombinant CRM197 protein, wherein the refolding is carried out by adding a refolding buffer containing Triton-X and 3-[(3-cholamidopropyl)dimethylammonio]-1-propanesulfonate (CHAPS).

In the present invention, a non-denaturing solubilization agent is used to solubilize CRM197 protein inclusion bodies, and thus, active CRM197 proteins can be produced with high yield.

### BRIEF DESCRIPTION OF THE DRAWINGS

FIG. 1 shows a plasmid map of pEThCRM.

FIG. 2 shows the SDS-PAGE analysis of soluble (S) and insoluble (I) fractions obtained from Escherichia coli transformed with pEThCRM (the arrow: CRM197).

FIG. 3 shows purification of CRM197 solubilized from inclusion bodies (M: marker, lane 1: soluble fraction, lane 2: insoluble fraction, lane 3 and 4: His-trap column product, lane 5: CRM197 purified by size exclusion chromatography).

FIG. 4 shows the procedure for recovery and purification of insoluble CRM197 proteins.

FIG. 5 shows the Western blot result for each fraction obtained by Ni-affinity chromatography.

FIG. 6 shows the result of ELISA analysis of standard CRM197 (Sigma) and CRM197 exhibiting the anti-DT activity.

FIG. 7 is the agarose (1%) gel electrophoresis showing the nuclease activity of CRM197.

FIG. 8 shows the result of binding activities of standard CRM197 and CRM197 obtained from the invention of the present application for HB-EGF as measured by sandwich ELISA.

FIG. 9 illustrates Table 1 which shows the investigation results to evaluate the recovery technique in accordance with the present invention.

### DETAILED DESCRIPTION

Hereinafter, preferred examples of the present invention are described in detail. When it is determined that describing relevant known techniques in detail during the course of describing the present invention can obscure the essence of the present invention, such detailed description will be excluded. Throughout the specification, when a particular part is said to “include” an element, the presence of other elements is not precluded and other elements may be further included, unless explicitly indicated otherwise.

The present invention discloses a method for producing a recombinant CRM197 protein, the method comprising: (a) a step for culturing a transformant transformed with an expression vector containing a gene encoding CRM197 protein; (b) a step for obtaining a culture of the transformant and disrupting the cell to recover an inclusion body; (c) a step for solubilizing the inclusion body; (d) a step for treating the solubilized inclusion body with a refolding reaction mixture and performing a refolding process; and (e) a step for purifying the refolded CRM197 protein by chromatography.

As used herein, the term “recombinant” microorganism refers to a microorganism that typically includes at least one exogenous nucleotide sequence, for example, in a plasmid or vector.

As used herein, the term “vector” refers to any nucleic acid including a competent nucleotide sequence, which is inserted into a host cell to be recombined with the genome of a host cell or to autonomously replicate within the host cell as a plasmid. Examples of the vector include linear nucleic acids, plasmids, phagemids, cosmids, and the like.

As used herein, the term “transformation” refers to the process by which an exogenous DNA enters the host cell in the presence or absence of an accompanying substance. The term “transfected cell” refers to a cell having exogenous DNA introduced into the cell. DNA can be introduced into the cell, so that the nucleic acid can be inserted into a chromosome or replicated as an extrachromosomal material.

As used herein, the term “host cell” may be an acceptor of any recombinant vector(s) or isolated polynucleotides of the invention, or includes an individual cell or a cell culture, which is the acceptor. The host cell may be a progeny of a single host cell, and the progeny do not have to be exactly the same as the original parent cell (in terms of form or total DNA complement) due to natural, accidental or artificial mutations and/or variations. The host cell includes a cell that has been transfected, transformed or infected with the recombinant vector or polynucleotide of the invention in vivo or in vitro.

In preparing the CRM197 protein of the present invention, first, i) a step for preparing a DNA sequence encoding the CRM197 protein may be performed. The DNA sequence encoding the CRM197 protein may be prepared by various methods known in the art such as chemical synthesis, RT-PCR, and the like. It is preferable to prepare the DNA sequence by PCR using the DNA of a target protein as a template and a primer capable of amplifying the target gene. The primer for amplifying the target gene may be prepared by a variety of methods, including, for example, cloning and restriction digestion of appropriate sequences, direct chemical synthesis by such as the phosphotriester method of Narang et al.; the diethylphosphoramidite method of Beaucage et al.; and the solid support method of U.S. Pat. No. 4,458,066.

The DNA sequence encoding the CRM197 protein may be a nucleotide sequence encoding the polypeptide set forth in SEQ ID NO: 1 or a nucleotide sequence complementary thereto.

[SEQ ID NO: 1]
MGSSHHHHHHSSGLVPRGSHMDDDDKGADDVVDSSKSFVMENFSSYHGT
KPGYVDSIQKGIQKPKSGTQGNYDDDWKEFYSTDNKYDAAGYSVDNENP
LSGKAGGVVKVTYPGLTKVLALKVDNAETIKKELGLSLTEPLMEQVGTE
EFIKRFGDGASRVVLSLPFAEGSSSVEYINNWEQAKALSVELEINFETR
GKRGQDAMYEYMAQACAGNRVRRSVGSSLSCINLDWDVIRDKTKTKIES
LKEHGPIKNKMSESPNKTVSEEKAKQYLEEFHQTALEHPELSELKTVTG
TNPVFAGANYAAWAVNVAQVIDSETADNLEKTTAALSILPGIGSVMGIA
DGAVHHNTEEIVAQSIALSSLMVAQAIPLVGELVDIGFAAYNFVESIIN
LFQVVHNSYNRPAYSPGHKTQPFLHDGYAVSWNTVEDSIIRTGFQGESG
HDIKITAENTPLPIAGVLLPTIPGKLDVNKSKTHISVNGRKIRMRCRAI
DGDVTFCRPKSPVYVGNGVHANLHVAFHRSSSEKIHSNEISSDSIGVLG
YQKTVDHTKVNSKLSLFFEIKS

Next, ii) a step for preparing a recombinant expression vector by introducing the DNA sequence so as to be operably fused to the expression vector may be performed.

Standard recombinant nucleic acid methods may be used to express the CRM197 protein of the invention. In one embodiment, a nucleic acid sequence encoding the CRM197 protein of the invention may be cloned into a nucleic acid expression vector, together with, e.g., appropriate signal and processing sequences and regulatory sequences for transcription and translation.

The recombinant expression vector for the CRM197 protein of the invention may include, for example, a regulatory sequence including a promoter operably linked to a sequence encoding the CRM197 protein of the invention. Non-limiting examples of inducible promoters that may be used include lactose inducible promoters, arabinose inducible promoters, bacteriophage lambda PL promoters, T7 phage-lac regulatory site conjugated promoters. This construct may be introduced into a suitable host cell, e.g., bacterial cell, yeast cell, insect cell, or tissue culture cell. A large number of suitable vectors and promoters are known to those skilled in the art, and are commercially available for generating the recombinant constructs of the present invention.

Known methods can be used to construct vectors containing the polynucleotide of the present invention and appropriate transcriptional/translational control signals. These methods include in vitro recombinant DNA techniques, synthetic techniques, and in vivo recombination/genetic recombination. See, techniques described in e.g., Sambrook & Russell, Molecular Cloning: A Laboratory Manual, 3rd Edition, Cold Spring Harbor Laboratory, N.Y. (2001) and Ausubel et al., Current Protocols in Molecular Biology (Greene Publishing Associates and Wiley Interscience, N.Y. (1989)).

Next, iii) a step for transforming the host with the recombinant expression vector may be performed.

A wide variety of E. coli host cells may be used to express DNA sequence of the CRM197 protein according to the present invention. These hosts include E. coli from which glutathione reductase and thioredoxin reductase were removed or E. coli from which genes related thereto were removed in combination, both for creating an oxidative environment in the cell, or E. coli from which related genes were removed to minimize the effects of endotoxins on purification (clear coli). Preferable host organisms include Escherichia coli Clear coli BL21, BL21 SHuffle, and the like.

Transformation or transfection into the host cell in the present invention includes any methods of introducing a nucleic acid into an organism, cell, tissue or organ, and may be performed by selecting a suitable standard technique depending on the host cell as known in the art. Such methods include, but are not limited to, electroporation, calcium phosphate (CaPO₄) precipitation, calcium chloride (CaCl₂) precipitation, transformation, and the like.

Next, the step for culturing the resulting transformant to express the DNA sequence may be performed. The host cells transformed according to the invention are cultivated in a nutrient medium suitable for the production of fusion proteins using known techniques. For example, the cells may be cultivated by small-scale or large-scale fermentation, shake flask cultivation in laboratory or industrial fermenters performed in a suitable medium and under conditions allowing the polypeptide to be expressed and/or isolated. The cultivation takes place in a suitable nutrient medium containing carbon and nitrogen sources and inorganic salts, using known techniques. Suitable media can be obtained from commercial suppliers or prepared according to the components and compositional ratios described in publications such as, for example, the catalog of the American Type Culture Collection. If the fusion protein is secreted directly into the medium, it can be separated directly from the medium, if the fusion protein is not secreted, it can be separated from the cell's lysate.

The next step of the method for preparing the CRM197 protein in the present invention is the step for disrupting the cells in the culture medium obtained above and recovering and washing the inclusion body. In this step, a cell pellet is obtained by centrifugation or filtration, and an eluate containing a cell lysate is obtained by removing media components including a ruptured cell wall or cell membrane. Specifically, lysis of the cell pellet may be performed by methods well-known in the art, such as the use of alkalis, surfactants (CHAPS, SDS, etc.), organic solvents and enzymes (lysozyme, etc.), sonication and the use of a high pressure crusher (French Press), periodic application of high temperature, pressure, freeze-thaw cycles, and the like. The clean eluate may be obtained by removing a minimal area of biomass in the cell lysate through cell fractionation. For example, the clean eluate may be obtained by taking a supernatant free of undisrupted parts of cells or insoluble cell debris through centrifugation. If necessary, the eluate may be subjected to other additional purification procedures known in the art. For example, the procedure of disrupting the cell may be performed using lysozyme. After breaking E. coli using lysozyme, the inclusion body is recovered through centrifugation and washed with a buffer containing a detergent several times to remove impurities.

The next step is the step for solubilizing the inclusion body.

In the present invention, the step for solubilizing the inclusion body may be performed by a non-denaturing solubilization method.

Conventionally, inclusion bodies are solubilized using high concentrations of denaturants and chaotropes like urea and guanidine hydrochloride. For proteins containing multiple cysteine residues, β-mercaptoethanol or dithiothreitol are added to these solubilization agents to reduce incorrect hydrolytic bonds. Solubilization of inclusion bodies using high concentrations of chaotropes results in complete disruption of protein structure, which frequently leads to aggregation of protein molecules during the refolding process. This can be a limiting factor when applying to proteins containing multiple cysteine residues, even though it may not be the case in all cases. Interestingly, the fact that inclusion bodies are dynamic in nature and exist as an equilibrium between folded and aggregated protein molecules was harnessed in solubilizing inclusion bodies under non-denaturing condition without assistance of any solubilization agent. According to amino acid sequencing, CRM197 has two internal disulfide bonds located between 186-201 and 461-471. Since CRM197 inclusion bodies are likely to have their original secondary structures, it may be advantageous to apply the non-denaturing solubilization method to recover correct disulfide bond of rCRM197 because they do not completely unfold these native protein structures unlike high concentrations of chaotropes.

In the present invention, the non-denaturing solubilization method is known as a mild solubilization method and in the present invention, for the non-denaturing solubilization method, see Singh et al., Microbial Cell Factories (2015) 14:41.

The non-denaturing solubilization method can improve the recovery of bioactive proteins by preventing the aggregation of the proteins during the refolding process while intactly maintaining the secondary structures of the proteins present in the inclusion bodies.

Preferably, the denaturing solubilization method may use Tris-Cl buffer, DMSO, n-propanol, or sarkosyl solution, more preferably, sarkosyl solution, and most preferably, 2% (w/v) or less sarkosyl solution.

Thereafter, the solubilized CRM197 protein may be refolded using a refolding buffer. Preferably the refolding step may be performed by adding a refolding buffer containing Triton X-100 and 3-[(3-cholamidopropyl)dimethylammonio]-1-propanesulfonate (CHAPS).

Thereafter, the step for purifying the refolded CRM197 protein may be performed.

The CRM197 protein may be purified in a conventional manner. The purification method is exemplified by salting out (e.g., ammonium sulfate precipitation, sodium phosphate precipitation), solvent precipitation (e.g., protein fractional precipitation using acetone, ethanol, and the like), dialysis, gel filtration, column chromatography such as ion exchange and reversed phase column chromatography, and ultrafiltration, and these methods may be performed alone or in combination. Most preferably, the CRM197 protein may be purified using nikel-nitrilotriacetic acid (Ni-NTA) affinity chromatography.

<Experimental Method>

1. Materials

The restriction endonucleases Ndel and HindIII, T4 DNA ligase, and EX taq polymerase were obtained from Takara (Kyoto, Japan). The expression vector pET28a (+) was obtained from Novagen (Darmstadt, Germany). The Clear coli BL21(DE3) (Lucigen, Wis.) was used for protein expression.

2. Gene Cloning and Expression of CRM197

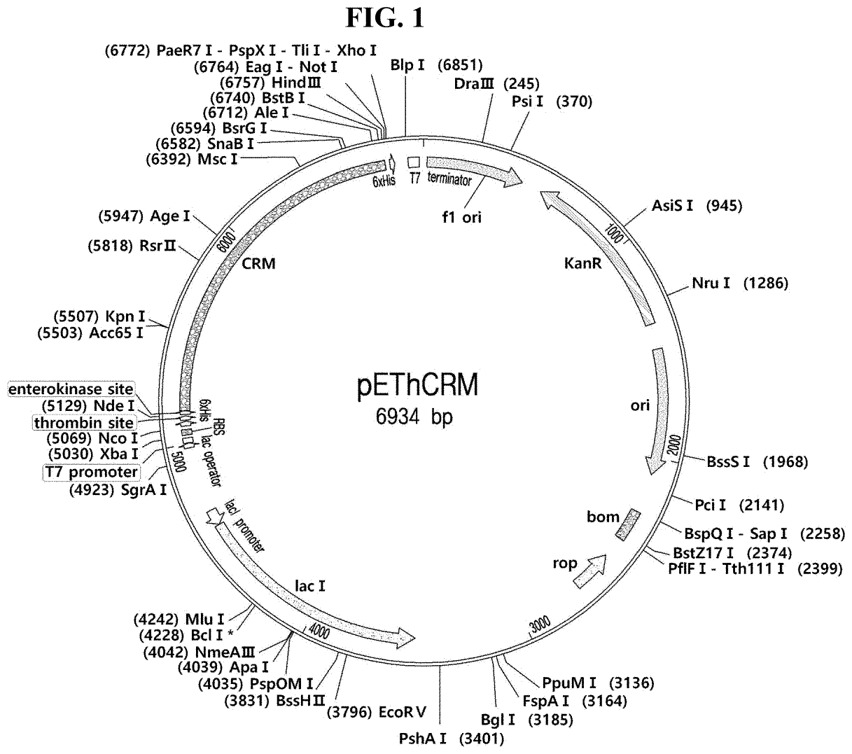

The synthetic gene corresponding to CRM197 (1,611 bp) was optimized for E. coli codon usage (GenScript, Piscataway, NJ) (S1 S1). The synthetic crm197 gene was cloned into pET28a(+) vector (Novagen) using Ndel and HindIII restriction sites. The gene was designed to include at the 5′ end, oligonucleotide sequences (66 bp) encoding a short histidine tag and enterokinase cleavage site. Cloning procedures were performed following standard techniques. The resulting pEThCRM plasmid (FIG. 1) was transformed into ClearColi BL21(DE3) (Lucigen) cells using the heat shock method, and transformants were selected on LB agar plate supplemented with kanamycin. The recombinant cells harboring pEThCRM were cultivated with shaking at 200 rpm, 37° C. with 30 pg/ml kanamycin until the OD600 reached 0.6. Isopropyl-β-d-thiogalactopyranoside (IPTG) was added to the culture medium at 0.5 mM to induce protein expression, and cultures were incubated at 37° C. for an additional 2 h.

3. Solubilization of Insoluble rCRM197

The harvested cells were resuspended in a 20 mM Tris-HCl buffer (pH 7.5) with protease inhibitor cocktail (Promega, Wis.), then disrupted using an ultra sonicator on ice. The insoluble inclusion bodies were separated from the cell lysate by centrifugation (13,000×g for 20 min at 4° C.). The pellet was carefully resuspended in 2 wt % or less N-Lauroylsarcosine sodium salt (sarkosyl) solution and then incubated at 4° C. until most of pellet was solubilized with gentle shaking. Solubilized samples were centrifuged at 13,000×g for 20 min at 4° C., and the supernatant was collected for the next purification step.

4. Purification of rCRM197

The folding of rCRM197 in the supernatant was preceded by drop-wise dilution into a tenfold volume of folding buffer (1% Triton X-100, 10mM CHAPS). Then, a tenfold volume of His-tag column equilibrating buffer (20 mM sodium phosphate buffer, 500 mM NaCl,) was added into the folding buffer, and the final pH of the solution was adjusted to 7.5. The Solubilized samples were loaded into a His-tagged affinity chromatography column (5-ml, HisTrap, Amersham biosciences, Little chalfont, England). Non-specific binding was removed by washing with 10 column volumes of the same equilibrating buffer, and the bound protein was eluted with the same buffer containing 250 mM imidazole at a flow rate of 1 ml/min by using a fast protein liquid chromatography (FPLC) system (Amersham biosciences) in a cold room. The resulting rCRM197 elution fraction was dialyzed with 20 mM Tris-HCl (pH 7.5) buffer at 4° C. using Slide-A-Lyzer dialysis cassette (10 kD MWCO, Thermo scientific). For additional purification, dialyzed fraction was concentrated by using micon ultra centrifugal filters (10 kD MWCO, Sigma-aldrich, MO). The resulting concentrated fraction was adapted to Sephacryl S-300 gel filtration column 16/60 (GE Healthcare, UK) (flow rate 0.6 ml/min). Subsequently, the existence and the purity of rCRM197 elution fraction were evaluated by SDS-PAGE followed by quantitative analysis tool (Image Lab Ver.5.2.1 build 11, Biorad) having gel image staind with Coomassie Brilliant Blue R250 staining solution (Biorad). The detection of target rCRM197 was achieved by western blot with murine monoclonal anti-diphtheria toxin (1:1,000; Abcam, Cambridge, England) as primary antibody and goat polyclonal anti-mouse-lgG conjugated to horseradish peroxidase (HRP) (1:2,500; Abcam) as secondary antibody.

5. Nuclease Activity Assay

For comparison commercial CRM197 and rCRM197, The nuclease activity was determined by incubating at 37° C. 2.5 μg of CRM197 with 500 ng of lambda DNA (Takara) with the proper reaction buffer (10 mM Tris-HCl pH 7.5, 2.5 mM CaCl₂, 2.5 mM MgCl₂) . The reaction was stopped by the addition of 5 mM EDTA with appropriate time interval. Samples were analyzed by 1% agarose gel electrophoresis in TAE buffer; gels were then stained with MaestroSafe Nucleic Acid stains (MaestroGen, Hsinchu, Taiwan) and analyzed under UV illumination.

6. ELISA Assays

Enzyme-linked immunosorbent assay (ELISA) was used to evaluate the binding of purified rCRM197 and standard CRM197 ([G1u52]-Diphtheria toxin from C. diphtheriae; Sigma-aldrich). ELISA plates were coated overnight at 4° C. with 50 μl/well of each protein in PBS (4 μg/ mL). Plates were then blocked at room temperature for 2 h with 0.5% bovine serum albumin (BSA) in PBS (PBS-BSA). After washing plates with PBS supplemented with 0.05% Tween 20 (PBST), purified rCRM197 and standard CRM197 samples serially diluted in PBS-BSA were added to the plates (50 pl/well). Plates were incubated for 1 h at room temperature and then washed with PBST. HRP-conjugated Murine monoclonal anti-diphtheria toxin (1:2,500; Abcam) in PBS-BSA was added to the plates (50 pl/ well). After 1 h of incubation at room temperature, plates were washed and then incubated with SigmaFast OPD HRP substrate (Sigma-aldrich) for 20 min. The reaction was quenched with 3N H2504, and the absorbance of the wells was measured at 490 nm.

7. HB-EGF Binding Assays

In the sandwich binding assay, ELISA plates were coated overnight at 4° C. with 50 μl/well of standard CRM197 and rCRM197 (8 μg/ mL) and blocked with PBS-BSA solution. Then, HB-EGF protein serially diluted in PBS-BSA, were added and incubated for 1 h at room temperature and then washed with PBST. Subsequently, anti-HB-EGF antibodies (4 μg/ mL; Abcam) were added and incubated at room temperature for 1 h. After the plates were washed, HRP-conjugated goat polyclonal anti-rabbit-lgG (1:2,500; Abcam) was added to the plate (50 μl/ well). After 1 h of incubation at room temperature, plates were washed and then incubated with SigmaFast OPD HRP substrate (Sigma-aldrich) for 20 min. The reaction was quenched with 3N H2SO4, and the absorbance of the wells was measured at 490 nm.

<Experimental Results>

1. Expression of Recombinant CRM197

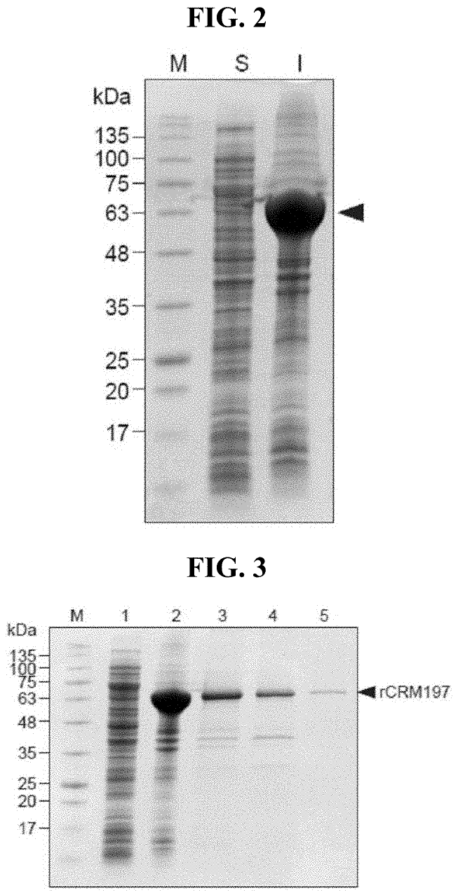

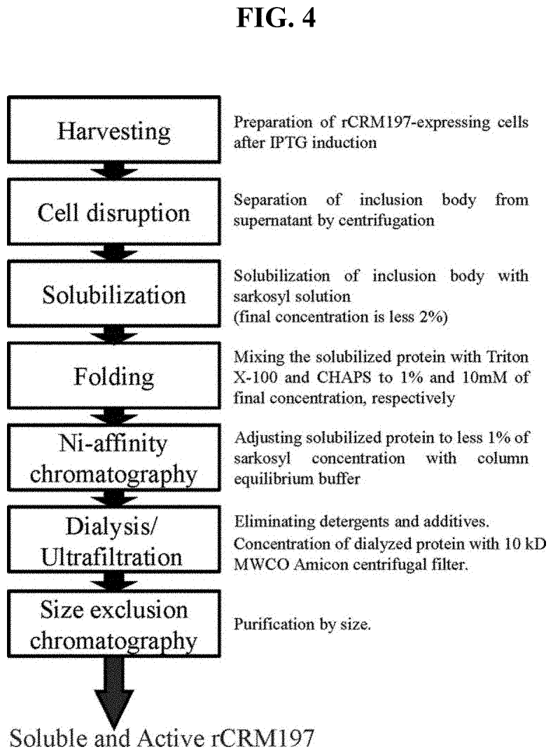

The synthetic nucleotide sequence of the CRM197 from C. diphtheriae, devoid of the natural signal sequence, was inserted into expression plasmid, pET28a (FIG. 1). The resulting plasmid, pEThCRM, permits expression of the synthetic CRM197 appended with an N-terminal his-tag and an enterokinase cleavage site for convenient purification. Following expression of the synthetic CRM197 in ClearColi BL21(DE3) cells, most of recombinant CRM197 (rCRM197) were detected at high levels in the insoluble fraction at 30° C. for 4 h with 0.5 mM IPTG, while a low amount of rCRM197 was produced as both soluble and insoluble forms under mild induction conditions (16° C. for 12 h) with 0.01-0.05 mM IPTG (FIGS. 2 and 3). Many researchers have attempted to produce CRM197 in E. coli by using a host with a more oxidizing cytoplasm, co-expressing chaperones, or secreting proteins into the periplasm. However, in the case of soluble expression in E. coli, the yield was not high enough for industrial scale production. Therefore, if the refolding yield of insoluble protein could be improved to industrial levels compared to conventional methods using strong denaturants (urea, guanidine-HCl), then our strategy allows cost-effective production applicable at industrial scales. This idea was tested by a mild refolding strategy using a non-denaturing detergent, which solubilized inclusion bodies but without full denaturation. FIG. 4 shows the overall procedure for recovery and purification of insoluble rCRM197 under non-denaturing conditions.

2. Solubilization and Purification of Insoluble rCRM197

In the case of rCRM197 insoluble expression, rCRM197 accumulated in the cytoplasm as an inclusion body when was expressed in the conventional induction condition as described. In order to solubilize the inclusion body, the insoluble fraction was separated from the clarified supernatant by centrifugation, then the pellet was carefully dissolved in non-denaturing solubilization agent, sarkosyl (N-Lauroylsarcosine), until the insoluble pellet almost disappeared as described in FIG. 4. It is thought that the separation of solubilized CRM197 from the insoluble debris is the most important step in obtaining rCRM197 with correct folding. The final concentration of sarkosyl was limited to 2% (w/v), since an increase of sarkosyl concentration can lead to unexpected aggregation of misfolded CRM197. After cell debris removal, the solubilized protein solution was adjusted to 1% or less sarkosyl concentration, followed by addition of 10 mM CHAPS and 1% (v/v) Triton X-100 for the next purification step. For Ni-affinity chromatography, the solubilized protein solution was adjusted with phosphate buffered saline. The inclusion bodies were solubilized with sarkosyl and successfully purified under native conditions using Ni-affinity chromatography and size-exclusion chromatography (FIG. 3).

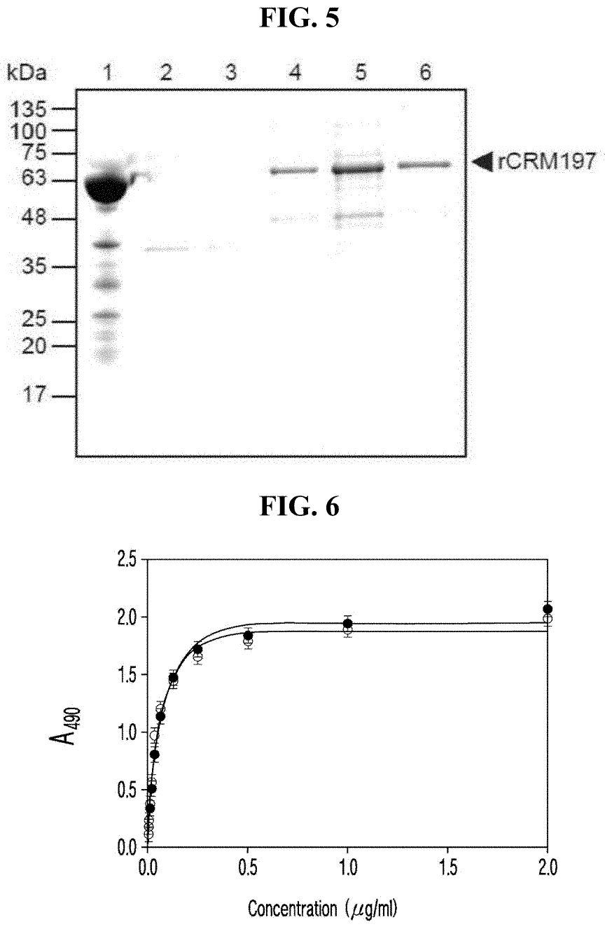

Following western blot analysis of Ni-affinity chromatography eluted samples, a strong rCRM197 band was detected in the purified fraction, while a low amount of unbound rCRM197 was also detected in the flow-through fraction (FIG. 5). This indicates that the amount of unfolded or misfolded rCRM197 was much lower than the recovered rCRM197. Even though it is not possible to say the exact yield for active rCRM197 at this point, most of the rCRM197 was expected to be solubilized and recovered through column chromatography (>85% of total rCRM197) under these much milder conditions, compared to strong denaturing conditions.

3. Recovery of Activity and Structure of rCRM197

We next investigated whether the purified rCRM197 could be fully recovered by our process described above. Our objective was to obtain high recovery yields of active CRM197 from insoluble rCRM197 expressed as inclusion bodies, while retaining correct structure and activity. To determine if purified rCRM197 permits immunodetection, we first performed ELISA with anti-Diphtheria toxin (DT) using Sigma standard CRM197 protein as control. As shown in FIG. 6, the binding affinity of anti-DT to purified rCRM197 was shown to be essentially indistinguishable from standard CRM197, which indicates that purified rCRM197 recovered activity and structure similar to active CRM197. However, this result was limited for proving the correct folding of rCRM197, since antibody binding region was not representative for the entire CRM197 structure.

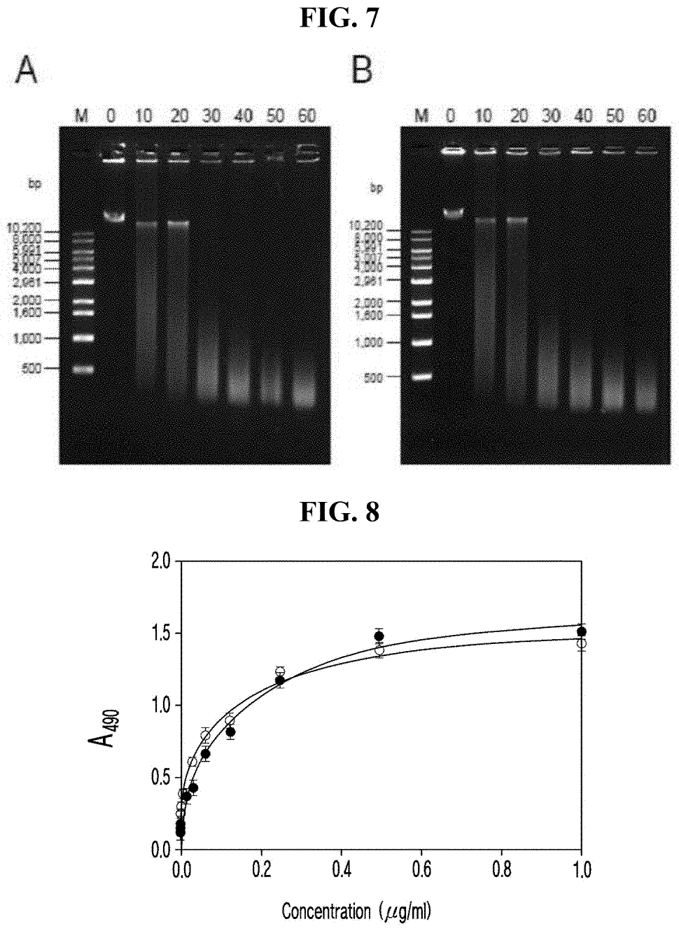

Hence, to demonstrate the recovery of active rCRM197, purified rCRM197 was additionally tested in a nuclease activity assay and in Heparin binding—epithermal growth factor (HB-EGF) binding assay. CRM197, like wild-type diphtheria toxin, possesses deoxyribonuclease activity, while diphtheria toxin possesses both protein degradation activity and deoxyribonuclease activity. It has been reported that maximum activity of CRM197 nuclease activity was detected at 37° C. As shown in FIG. 7, the purified rCRM197 appeared to possess almost the same activity at 37° C. as standard CRM197 (Sigma) devoid of an additional N-terminal polyhistidine-tag. Likewise, Stefan et al. also reported that the N-terminal His tag did not interfere with the biochemical activity of their CRM197 in vitro.

CRM197 has been reported to bind to the soluble form of HB-EGF which is highly expressed in some cancers. Taking advantage of this feature, the binding ability of CRM197 to HB-EGF was examined to determine if CRM197 was correctly folded. We performed the HB-EGF binding assay by a sandwich ELISA method. As shown in FIG. 8, the sandwich ELISA confirmed that purchased HB-EGF bound strongly to immobilized rCRM197 as well as standard CRM197 (Sigma). The HB-EGF binding assay for rCRM197 prepared from inclusion bodies also confirmed that rCRM197 appeared to be purified correctly folded, despite having less purity than the standard CRM197. Taken together, these results indicate that rCRM197 is correctly folded and retains the same nuclease activity as standard CRM197 (Sigma), consistent with the above ELISA data using anti-DT.

4. Comparison of Production Yield

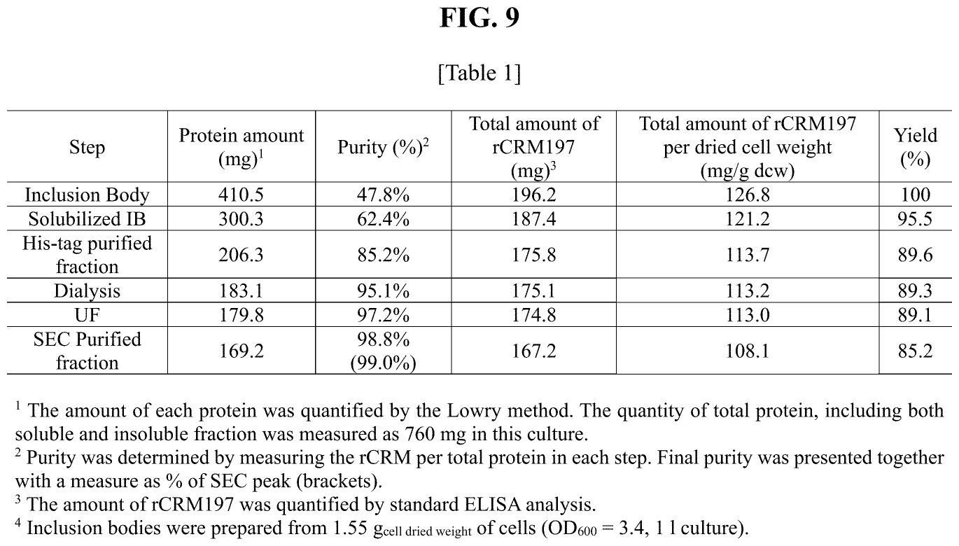

To evaluate our recovery technique, we investigated the overall purification yield from the preparation step of inclusion bodies (see FIG. 9, Table 1). Our recovery yield of active rCRM197 from inclusion bodies was appeared to reach up to about 85%. Additionally, almost all purified rCRM197 were active form as shown in our nuclease activity assay and HB-EGF binding assay (FIGS. 7 and 8).

In this study, our recovery yield of rCRM197 indicates that our technology has great potential considering that the protein quantity of inclusion body in E. coli can reach up to 25.8% of the total protein (Table 1). It means that the production of rCRM197 could be improved efficiently if the quantity of total rCRM197 were increased by the optimization of culture condition, regardless of soluble or insoluble.

Above, exemplary embodiments of the present invention have been described in detail with reference to the drawings. Descriptions of the present invention are merely exemplary, and it is to be understood that the present inventions could be easily modified into different specific forms by a person with ordinary skill in the art, without changing the technical concept or essential properties of the present invention.

Therefore, the scope of the present invention is not specified by the detailed description, but rather by the claims disclosed below. All modifications or modified forms derived from the meaning, scope, and equivalent concepts of the claims are to be construed as being within the scope of the present invention.

1. A method for producing a recombinant CRM197 protein, comprising:
(a) a step for culturing a transformant transformed with an expression vector containing a gene encoding CRM197 protein;
(b) a step for obtaining a culture of the transformant and disrupting the cell to recover an inclusion body;
(c) a step for solubilizing the inclusion body;
(d) a step for treating the solubilized inclusion body with a refolding reaction mixture and performing a refolding process; and
(e) a step for purifying the refolded CRM197 protein by chromatography.

2. The method for producing a recombinant CRM197 protein of claim 1, wherein the solubilization is performed by a non-denaturing solubilization method.

3. The method for producing a recombinant CRM197 protein of claim 2, wherein the non-denaturing solubilization method uses Tris-Cl buffer, DMSO, n-propanol or sarkosyl solution.

4. The method for producing a recombinant CRM197 protein of claim 1, wherein the refolding is carried out by adding a refolding buffer containing Triton-X and 3-[(3-cholamidopropyl)dimethylammonio]-1-propanesulfonate (CHAPS).
